(19) United States
(12) Patent Application Publication
(10) Pub. No.: US 2004/0185446 A1
(43) Pub. Date: Sep. 23, 2004

(54) Cpn60 targets for quantification of microbial species

(76) Inventors: Alison M. Jones, Eden Prairie, MN (US); W. Wade Robey, Excelsior, MN (US); Sean Hemmingsen, Saskatoon (CA); Janet Hill, Saskatoon (CA); Andrew Van Kessel, Saskatoon (CA)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/392,387

(22) Filed: Mar. 18, 2003

(51) Int. Cl. C12Q 1/68; C12P 19/34

(52) U.S. Cl. 435/6; 435/91.2

(57) ABSTRACT

cpn60 nucleic acid-based methods for determining microbial profiles are provided, as are cpn60 primers and probes for use in methods of the invention, and kits containing such primers and probes.

### TECHNICAL FIELD

This invention relates to determining microbial profiles, and more particularly to determining microbial profiles based on detection and quantification of cpn60 nucleic acids from various microbial species present within a sample.

### BACKGROUND

Microbial profiles are representations of individual strains, subspecies, species, and/or genera of microorganisms within a community of microorganisms. Generally, determining a microbial profile involves taxonomic and/or phylogenetic identification of the microbes in a community. A microbial profile also can include quantitative information about one or more members of the community. Once one or more microorganisms have been identified in a microbial community, microbial profiles can be presented as, for example, lists of microorganisms, graphical or tabular representations of the presence and/or numbers of microorganisms, or any other appropriate representation of the diversity and/or population levels of the microorganisms in a community. Microbial profiles are useful for identifying pathogenic and non-pathogenic microbial organisms in biological and non-biological samples (e.g., samples from animals, the environment, or inanimate objects).

A microbial profile can be determined using any of a number of methods. For example, the microbes in a sample can be cultured and colonies identified and/or enumerated. It has been estimated, however, that culturing typically recovers only about 0.1% of the microbial species in a sample (based on comparisons between direct microscopic counts and recovered colony-forming units). An improvement on culture-based methods is a community-level physiological profile. Such a profile can be determined by monitoring the capacity of a microbial community to utilize a particular carbon source, with subsequent detection of the end product of metabolism of the carbon source. Profiling the physiology of a microbial community can yield qualitative and semi-quantitative results.

Culture-independent methods to determine microbial profiles can include extracting and analyzing microbial macromolecules from a sample. Useful target molecules typically include those that as a class are found in all microorganisms, but are diverse in their structures and thereby reflect the diversity of the microbes. Examples of target molecules include phospholipid fatty acids (PLFA), polypeptides, and nucleic acids. PLFA analysis is based on the universal presence of modified fatty acids in microbial membranes, and is useful as a taxonomic tool. PLFAs are easily extracted from samples, and separation of the various signature structures reveals the presence and abundance of classes of microbes. This method requires appropriate signature molecules, which often are not known or may not be available for the microbes of interest. In addition, the method requires that an organism's PLFA content does not change under different metabolic conditions. Another limitation to using PLFAs as target molecules is that widely divergent organisms may have the same signature set of PLFAs.

Other less direct measures can be made that can provide insight into changes that might be taking place in the microbial profile within a particular environment. For example, pathogenic changes in the gastrointestinal tract (GIT) microbial profile of an animal may lead to morphometric changes in GIT structure. These morphometric changes can be measured by, for sample, excising GIT tissues and histologically evaluating for the number, size, shape, mucosal-cell turnover, and condition of the villi. The microscopic appearance of the villi can correlate with the microbial ecology of the animal, as many of the resident organisms attach directly to the mucosa and can cause damage and/or destruction of the absorptive surface.

Techniques such as immunohistochemical analysis also can be employed as indicative measures of pathogenic microbes in animal tissues. The presence of circulating leukocytic cytokines (lymphokines and monokines), as well as the presence of immunoglobulins (e.g., IgM, IgG, or IgA) either in the systemic circulation or localized in a tissue at the site of an antigenic insult can be correlated with the presence of potentially deleterious microbes.

Various nucleic acid-based assays also can be employed to determine a microbial profile. Some nucleic acid-based population methods use, for example denaturation and reannealing kinetics to derive an indirect estimate of the guanine and cytosine (% G+C) content of the DNA in a sample. The % G+C technique provides an overall view of the microbial community, but typically is sensitive only to massive, changes in the make-up of the community.

Genetic fingerprinting also can be used to determine a microbial profile. Genetic fingerprinting utilizes random-sequence oligonucleotide primers that hybridize specifically to random sequences throughout the genome. Amplification results in a multitude of products, and the distribution of these products is referred to as a genetic fingerprint. Particular patterns can be associated with a community of microbes in the sample. Genetic fingerprinting, however, lacks the ability to conclusively identify specific microbial species.

Denaturing or temperature gradient gel electrophoresis (DGGE or TGGE) is another technique that can be used to determine a microbial profile. As amplification products are electrophoresed in gradients with increasing denaturant or temperature, the double-stranded molecule melts and its mobility is reduced. The melting behavior is determined by the nucleotide sequence, and unique sequences will resolve into individual bands. Thus, a D/TGGE gel yields a genetic fingerprint characteristic of the microbial community, and the relative intensity of each band reflects the abundance of the corresponding microorganism. An alternative format includes single-stranded conformation polymorphism (SSCP). SSCP relies on the same physical basis as % G+C renaturation methods, but reflects a significant improvement over such methods.

In addition, a microbial profile can be determined using terminal restriction fragment length polymorphism (TRFLP) analysis. Amplification products can be analyzed for the presence of known sequence motifs using restriction endonucleases that recognize and cleave double-stranded nucleic acids at these motifs. For example, the enzyme HhaI cuts at 5′-GCGC-3′ sites. Amplification products can be tagged at one end with a fluorescently labeled primer and digested with HhaI. Resolution of the digest by electrophoresis will yield a series of fluorescent bands with lengths determined by how far a 5′-GCGC-3′ motif lies from the terminal tag. The principal advantages of TRFLP are its robustness and its low cost. Unlike D/TGGE, experimental conditions need not be stringently controlled since the profiles are size-based and thus can be generated by a variety of gel systems, including automated DNA sequencing machines. Alternative approaches include “amplified ribosomal DNA restriction analysis (AADRA)” in which the entire amplification product, rather than just the terminal fragment, is considered. AADRA, however, becomes unmanageable with communities containing many species.

A microbial profile also can be determined by cloning and sequencing microbial nucleic acids present in a biological or non-biological sample. Cloning of individual nucleic acids into Escherichia coli and sequencing each nucleic acid gives the highest density of information but requires the most effort. Although sequencing of nucleic acids is an automated process, routine monitoring of changes in the microbial profile of an animal by cloning and sequencing nucleic acids from the microorganisms still requires considerable time and effort.

Genotyping of 16S ribosomal DNA (rDNA) is another way to determine a microbial profile. 16S rDNA sequences are universal and are composed of both (1) highly conserved regions, which allow for design of common amplification primers, and (2) open reading frame (ORF) regions containing sequence variations, which allow for phylogenetic differentiation. 16S ribosomal sequences are relatively abundant in the RNA form. In addition to amplification using oligonucleotide primers, genotyping of 16S rDNA can be performed using other methods including restriction fragment length polymorphism (RFLP) analysis with Southern blotting.

Despite the existence of numerous methods for determining microbial profiles, a method that is rapid, sensitive, and quantitative would have significant utility.

### SUMMARY

The invention provides cpn60 nucleic acid-based methods that can be used to determine a microbial profile of a sample. Methods of the invention are very rapid and extremely sensitive, and can be used to detect the presence or absence of cpn60-containing microbes in general, as well as to identify what species of microbes are present and in what amounts. Using cpn60 primers and probes, methods of the invention can include amplifying cpn60 nucleotide sequences and detecting amplification products using techniques such as fluorescence resonance energy transfer (FRET). Primers and probes for detecting cpn60-containing microbial species also are provided by the invention, as are kits containing such primers and probes.

In one aspect, the invention features a method for quantifying the amount of one or more microbial species in a biological or non-biological sample. The method can include (a) providing the sample; (b) subjecting the sample to amplification in the presence of cpn60 primers, thereby generating an amplification product if a microbial species containing cpn60 is present in the sample; and (c) quantifying the amplification product, wherein the amount of the product is correlated with the amount of the microbial species in the sample.

The primers can be universal cpn60 primers and the quantifying can include hybridization of one or more species-specific cpn60 probes to the amplification product. The hybridization can be detected in real time. The correlation can employ a standard curve of known amounts of the microbial species. The one or more species-specific cpn60 probes can be differentially labeled. Alternatively, the primers can be universal cpn60 primers and the quantifying can include hybridization of a universal cpn60 probe to the amplification product.

The quantifying can include hybridization of a first cpn60 probe and a second cpn60 probe to the amplification product. The first cpn60 probe can be labeled with a donor fluorescent moiety (e.g., fluorescein), the second cpn60 probe can be labeled with a corresponding acceptor fluorescent moiety (e.g., LC-Red 640, LC-Red 705, Cy5, or Cy5.5), and the first and second cpn60 probes can hybridize to the amplification product in a manner such that fluorescence resonance energy transfer occurs.

The quantifying can include hybridization of one cpn60 probe to the amplification product. The cpn60 probe can be labeled with a donor fluorescent moiety and a corresponding acceptor fluorescent moiety. The cpn60 probe can have a nucleotide sequence that permits secondary structure formation, where the secondary structure formation results in spatial proximity between the first and second fluorescent moieties.

The quantifying can include measuring the interaction of a fluorescent dye with the amplification product. The interaction can be intercalation.

The sample can be selected from the group consisting of a biological tissue, a biological fluid, a biological elimination product, a water sample, a soil sample, and a swab from an inanimate object. The one or more microbial species can belong to genera selected from the group consisting of Escherichia, Salmonella, Campylobacter, Staphyococcus, Clostridium, Pseudomonas, Bifidobacterium, Bacillus, Enterococcus, Acanthamoeba, Cryptosporidium, Tetrahymena, Aspergillus, Candida, and Saccharomyces.

In another aspect, the invention features an article of manufacture containing one or more cpn60 primers and one or more cpn60 probes, and instructions for using the one or more cpn60 primers and one or more cpn60 probes for quantifying the amount of one or more microbial species in a biological or non-biological sample.

The invention also features a method for quantifying the amount of a particular microbial species in a biological or environmental sample. The method can include (a) providing the sample; (b) subjecting the sample to amplification in the presence of primers specific to the cpn60 gene of the microbial species, thereby generating an amplification product if the microbial species is present in the sample; and (c) quantifying the amplification product, wherein the amount of the product is correlated with the amount of the microbial species in the sample. The quantifying can include hybridization of a first cpn60 probe and a second cpn60 probe to the amplification product. The first cpn60 probe can be labeled with a donor fluorescent moiety (e.g., fluorescein), the second cpn60 probe can be labeled with a corresponding acceptor fluorescent moiety (e.g., LC-Red 640, LC-Red 705, Cy5, or Cy5.5), and the first and second cpn60 probes can hybridize to the amplification product in a manner such that fluorescence resonance energy transfer occurs.

The quantifying can include hybridization of one cpn60 probe to the amplification product. The cpn60 probe can be labeled with a donor fluorescent moiety and a corresponding acceptor fluorescent moiety. The cpn60 probe can contain a nucleotide sequence that permits secondary structure formation, where the secondary structure formation results in spatial proximity between the first and second fluorescent moieties.

The quantifying can include interaction of a fluorescent dye with the amplification product. The interaction can be intercalation.

The sample can be selected from the group consisting of a biological tissue, a biological fluid, a biological elimination product, a water sample, a soil sample, and a swab from an inanimate object. The microbial species can belong to a genera selected from the group consisting of Escherichia, Salmonella, Campylobacter, Staphyococcus, Clostridium, Pseudomonas, Bifidobacterium, Bacillus, Enterococcus, Acanthamoeba, Cryptosporidium, Tetrahymena, Aspergillus, Candida, and Saccharomyces.

In another aspect, the invention features a method for quantifying the amount of Clostridium perfringens in a biological or non-biological sample. The method can include (a) providing the sample; (b) subjecting the sample to amplification in the presence of cpn60 primers, thereby generating an amplification product if C. perfringens is present in the sample; and (c) quantifying the amplification product by hybridizing a cpn60 probe to the product, wherein the amount of the amplification product is correlated with the amount of C. perfringens in the sample. The cpn60 primers can have the nucleotide sequences set forth in SEQ ID NO:8 and SEQ ID NO:9. The cpn60 probe can have the nucleotide sequence set forth in SEQ ID NO:16.

In another aspect, the invention features a method for quantifying the amount of Salmonella enterica in a biological or non-biological sample. The method can include (a) providing the sample; (b) subjecting the sample to amplification in the presence of cpn60 primers, thereby generating an amplification product if S. enterica is present in the sample; and (c) quantifying the amplification product by hybridizing a cpn60 probe to the product, wherein the amount of the amplification product is correlated with the amount of S. enterica in the sample. The cpn60 primers can have the nucleotide sequences set forth in SEQ ID NO:10 and SEQ ID NO:11. The cpn60 probe can have the nucleotide sequence set forth in SEQ ID NO:17.

In another aspect, the invention features a method for quantifying the amount of Campylobacter jejuni in a biological or non-biological sample. The method can include (a) providing the sample; (b) subjecting the sample to amplification in the presence of cpn60 primers, thereby generating an amplification product if C. jejuni is present in the sample; and (c) quantifying the amplification product by hybridizing a cpn60 probe to the product, wherein the amount of the amplification product is correlated with the amount of C. jejuni in the sample. The cpn60 primers can have the nucleotide sequences set forth in SEQ ID NO:12 and SEQ ID NO:13. The cpn60 probe can have the nucleotide sequence set forth in SEQ ID NO:18.

In another aspect, the invention features a method for quantifying the amount of Escherichia coli in a biological or non-biological sample. The method can include (a) providing the sample; (b) subjecting the sample to amplification in the presence of cpn60 primers, thereby generating an amplification product if E. coli is present in the sample; and (c) quantifying the amplification product by hybridizing a cpn60 probe to the product, wherein the amount of the amplification product is correlated with the amount of E. coli in the sample. The cpn60 primers can have the nucleotide sequences set forth in SEQ ID NO:14 and SEQ ID NO:15. The cpn60 probe can have the nucleotide sequence set forth in SEQ ID NO:19.

In yet another aspect, the invention features an article of manufacture containing cpn60 primers having the nucleotide sequences set forth in SEQ ID NO:8 and SEQ ID NO:9, and a cpn60 probe having the nucleotide sequence set forth in SEQ ID NO:16. The article of manufacture can further contain instructions for using the primers and probes to quantify the amount of C. perfringens in a biological or non-biological sample.

The invention also features an article of manufacture containing cpn60 primers having the nucleotide sequences set forth in SEQ ID NO:10 and SEQ ID NO:11, and a cpn60 probe having the nucleotide sequence set forth in SEQ ID NO:17. The article of manufacture can further contain instructions for using the cpn60 primers and probes to quantify the amount of S. enterica in a biological or non-biological sample.

In another aspect, the invention features an article of manufacture containing cpn60 primers having the nucleotide sequences set forth in SEQ ID NO:12 and SEQ ID NO:13, and a cpn60 probe having the nucleotide sequence set forth in SEQ ID NO:18. The article of manufacture can further contain instructions for using the cpn60 primers and probes to quantify the amount of C. jejuni in a biological or non-biological sample.

In another aspect, the invention features an article of manufacture containing cpn60 primers having the nucleotide sequences set forth in SEQ ID NO:14 and SEQ ID NO:15, and a cpn60 probe having the nucleotide sequence set forth in SEQ ID NO:19. The article of manufacture can further contain instructions for using the cpn60 primers and probes to quantify the amount of E. coli in a biological or non-biological sample.

The invention also features isolated nucleic acids, e.g., primers and probes that contain the nucleotide sequences set forth in SEQ ID NOS:8-19. Such isolated nucleic acids can be of any length useful for primers and probes, e.g., 12, 13, 14, 15, 16, 17, 18, 19, 20, 21, 22, 23, 24, 25, 26, 27, 28, 29, or 30 nucleotides in length. Longer nucleotide sequences (e.g., 50, 100, 200, 500, or 1000 nucleotides in length) also can be useful in selected circumstances.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is the sequence of a cpn60 gene from Clostridium perfringens (SEQ ID NO:1; GenBank® Accession No. NC_{—}003366). Sequences to which the universal cpn60 primers described herein can hybridize (or the complement thereof) are underlined.

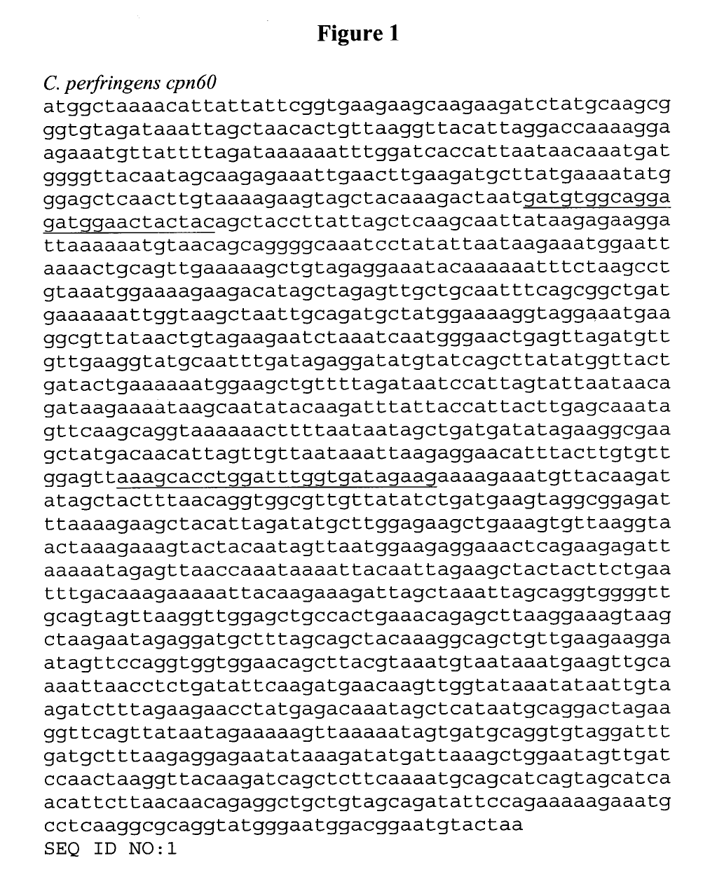

FIG. 2 is the sequence of a cpn60 gene from Escherichia coli (SEQ ID NO:2; GenBank® Accession No. NC_{—}000913). Sequences to which the universal cpn60 primers described herein can hybridize (or the complement thereof) are underlined.

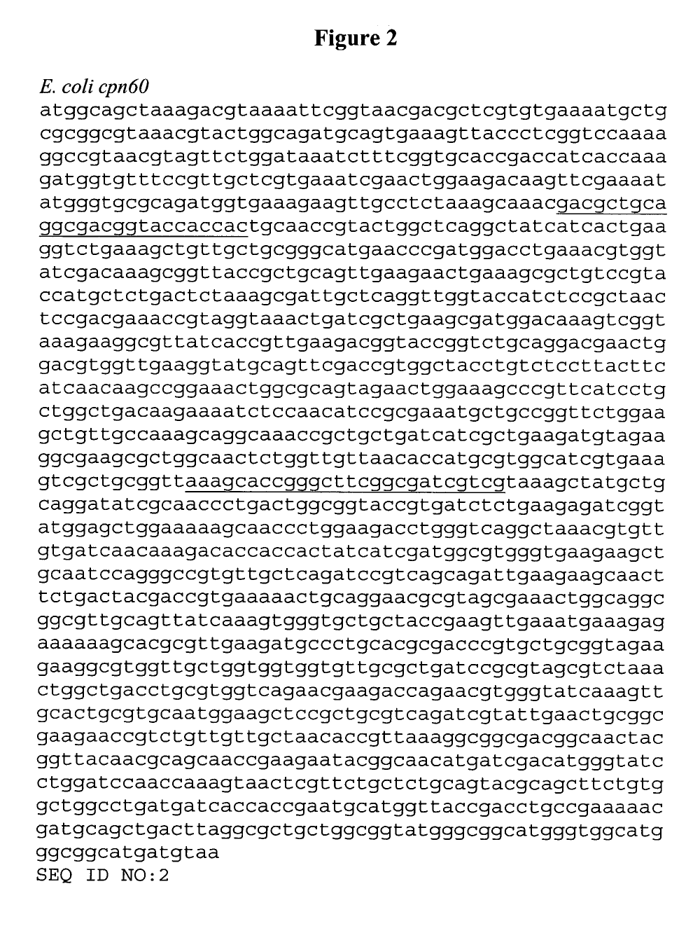

FIG. 3 is the sequence of a cpn60 gene from Staphylococcus coelicolor (SEQ ID NO:3; GenBank® Accession No. AL939121). Sequences to which the universal cpn60 primers described herein can hybridize (or the complement thereof) are underlined.

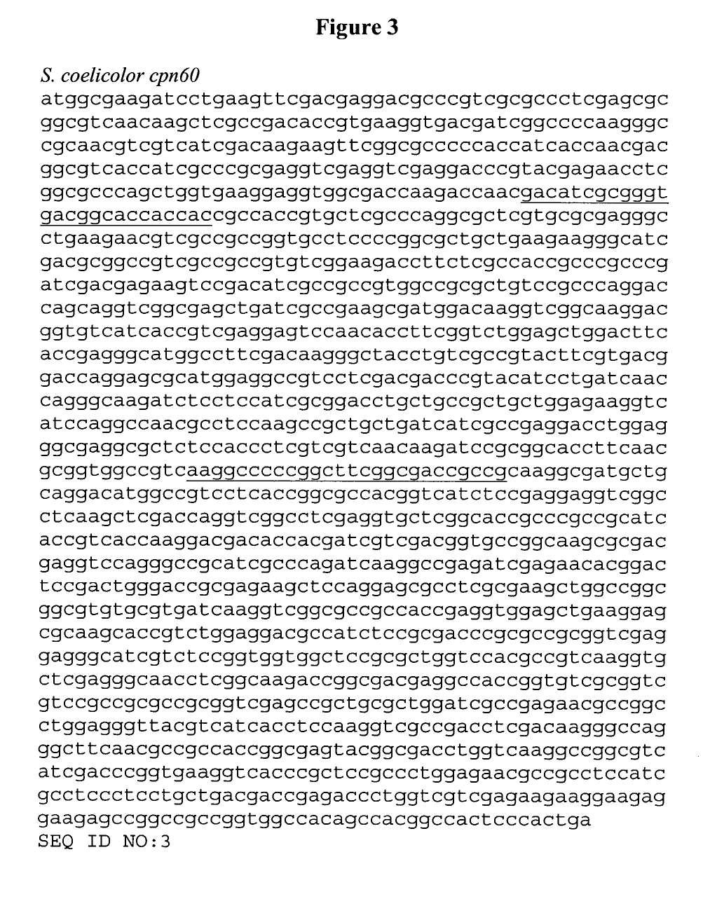

FIG. 4 is the sequence of a cpn60 gene from Campylobacter jejuni (SEQ ID NO:4; GenBank® Accession No. NC_{—}002163). Sequences to which the universal cpn60 primers described herein can hybridize (or the complement thereof) are underlined.

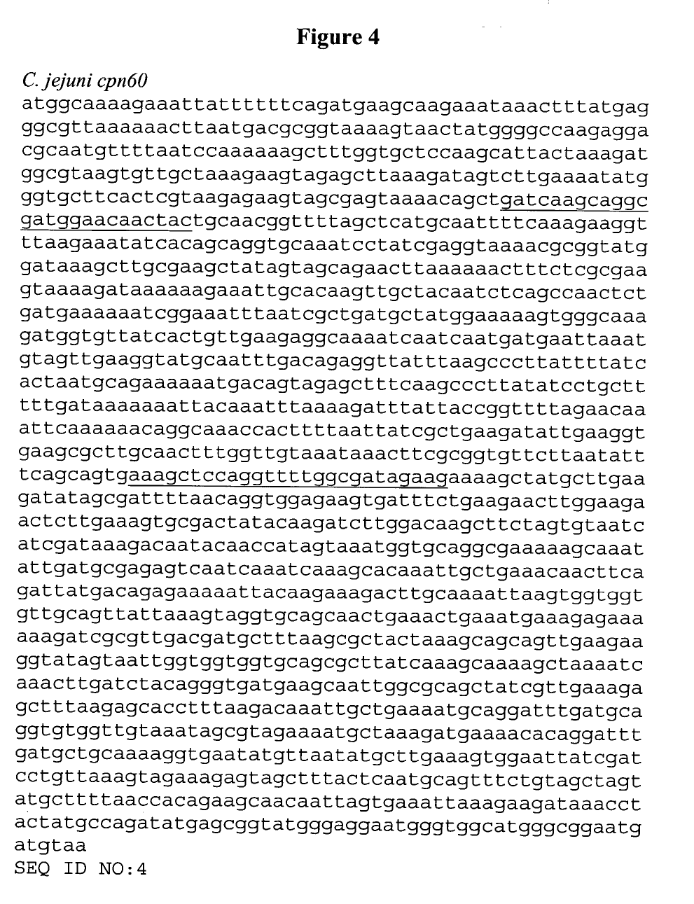

FIG. 5 is the sequence of a cpn60 gene from Salmonella enterica (SEQ ID NO:5; GenBank® Accession No. NC_{—}003198). Sequences to which the universal cpn60 primers described herein can hybridize (or the complement thereof) are underlined.

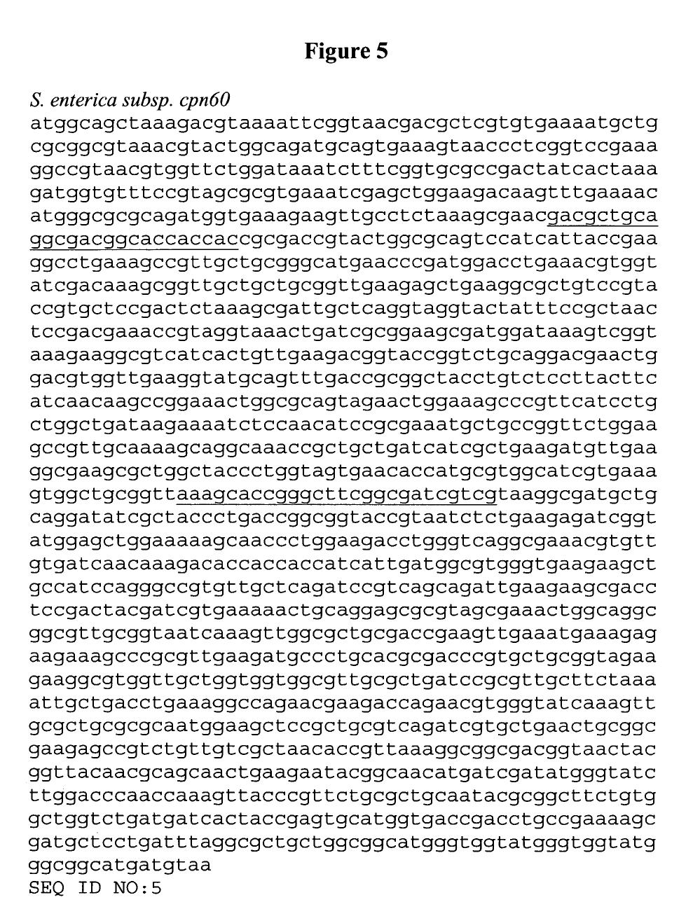

### DETAILED DESCRIPTION

Quantification of microbial organisms, including quantitative forms of microbial profiles, can be determined using methods that involve detection of cpn60 nucleic acid molecules. Methods of the invention are very rapid and extremely sensitive, and can be used to qualitatively and quantitatively detect cpn60-containing microbes. In addition to detecting and quantifying the amounts of individual microbial species within a sample, methods provided herein also use cpn60 to detect and quantitate the overall microbial load within a sample. Using cpn60 primers and probes, methods of the invention can include amplifying cpn60 nucleotide sequences using, for example, real-time polymerase chain reaction (PCR), and detecting amplification products with FRET. The invention provides primers and probes for detecting cpn60-containing microbial species, as well as methods for using such primers and probes to quantify the amount of one or more microbial species in a sample, and kits containing such primers and probes.

As used herein, “microbes” refer to bacteria, protozoa, and fungi. Microbial communities for which a microbial profile can be generated include, without limitation, prokaryotic genera such as Staphylococcus, Streptococcus, Pseudomonas, Escherichia, Bacillus, Brucella, Chlamydia, Clostridium, Shigella, Mycobacterium, Agrobacterium, Bartonella, Borellia, Bradyrhizobium, Ehrlichia, Haemophilus, Helicobacter, Heliobacter, Lactobacillus, Neisseria, Rhizobium, Streptomyces, Synechococcus, Zymomonas, Synechocyotis, Mycoplasma, Yersinia, Vibrio, Burkholderia, Franciscella, Legionella, Salmonella, Bifidobacterium, Enterococcus, Enterobacter, Citrobacter, Bacteroides, Prevotella, Xanthomonas, Xylella, and Campylobacter; protozoa genera such as Acanthamoeba, Cryptosporidium, and Tetrahymena; and fungal genera such as Aspergillus, Colletrotrichum, Cochliobolus, Helminthosporium, Microcyclus, Puccinia, Pyricularia, Deuterophoma, Monilia, Candida, and Saccharomyces.

Quantitative information about microbial levels can be obtained from various samples. As used herein, “biological sample” refers to any sample obtained, directly or indirectly, from a subject animal or control animal. Representative biological samples that can be obtained from an animal include or are derived from biological tissues, biological fluids, and biological elimination products (e.g., feces). Biological tissues can include biopsy samples or swabs of the biological tissue of interest, e.g., nasal swabs, throat swabs, or dermal swabs. The tissue can be any appropriate tissue from an animal, such as a human, cow, pig, horse, goat, sheep, dog, cat, bird, monkey, fish, clam, oyster, mussel, lobster, shrimp, and crab. Depending on the microbial organism, the tissue of interest to sample (e.g., by biopsy or swab) can be, for example, an eye, a tongue, a cheek, a hoof, a beak, a snout, a foot, a hand, a mouth, a teat, the gastrointestinal tract, a feather, an car, a nose, a mucous membrane, a scale, a shell, the fur, or the skin.

Biological fluids can include bodily fluids (e.g., urine, milk, lachrymal fluid, vitreous fluid, sputum, cerebrospinal fluid, sweat, lymph, saliva, semen, blood, or serum or plasma derived from blood); a lavage such as a breast duct lavage, lung lavage, a gastric lavage, a rectal or colonic lavage, or a vaginal lavage; an aspirate such as a nipple or teat aspirate; a fluid such as a cell culture or a supernatant from a cell culture; and a fluid such as a buffer that has been used to obtain or resuspend a sample, e.g., to wash or to wet a swab in a swab sampling procedure. Biological samples can be obtained from an animal using methods and techniques known in the art. See, for example, Diagnostic Molecular Microbiology: Principles and Applications (Persing et al. (eds.), 1993, American Society for Microbiology, Washington D.C.), hereby incorporated by reference in its entirety.

Biological samples also can be obtained from the environment (e.g., air, water, or soil). Methods are known for extracting biological samples (e.g., cells) from such samples. Additionally, a biological sample suitable for use in the methods of the invention can be a substance that one or more animals have contacted. For example, an aqueous sample from a water bath, a chill tank, a scald tank, or other aqueous environments with which a subject or control animal has been in contact, can be used in the methods of the invention to evaluate a microbial profile. A soil sample that one or more subject or control animals have contacted, or on which an animal has deposited fecal or other biological material, also can be used in the methods of the invention. For example, nucleic acids can be isolated from such biological samples using methods and techniques known in the art. See, for example, Diagnostic Molecular Microbiology: Principles and Applications (supra).

Methods of the present invention also can be used to detect the presence of microbial pathogens in or on non-biological samples. For example, a fomite may be sampled to detect the presence or absence of a microbial organism. A fomite is a physical (inanimate) object that serves to transmit, or is capable of transmitting, an infectious agent, e.g., a microbial pathogen, from animal to animal. (It is noted that inanimate objects such as food, air, and liquids are not considered fomites, but are considered infectious “vehicles,” or media that are routinely taken into the body.) Indeed, one study that evaluated the presence of Salmonella spp., Listeria spp., and Yersinia spp. pathogenic microbes on various abbatoir fomites detected Salmonella spp. on 11.1% of meat cleavers, 6.25% of worktables, and 5.6% of floors; Yersinia enterocolitica was found on 16.7% of slaughter floors and on 12.5% of worktables; and Listeria monocytogenes was isolated from 13.3% of cold room floor swabs and on 7.1% of hand-wash basins. See Kathryn Cooper, Guelph Food Technology Centre, “The Plant Environment Counts: Protect your Product through Environmental Sampling,” Meat & Poultry, May 1999, hereby incorporated by reference in its entirety. Nonlimiting examples of fomites include utensils, knives, drinking glasses, food processing equipment, cutting surfaces, cutting boards, floors, ceilings, walls, drains, overhead lines, ventilation systems, waste traps, troughs, machines, toys, storage boxes, toilet seats, door handles, clothes, gloves, bedding, combs, shoes, changing tables (e.g., for diapers), diaper bins, toy bins, food preparation tables, food transportation vehicles (e.g., rail cars and shipping vessels), gates, ramps, floor mats, foot pedals of vehicles, sinks, washing facilities, showers, tubs, buffet tables, surgical equipment and instruments, and analytical instruments and equipment.

A microbial organism may be left as a residue on a fomite. In such cases, it is important to detect accurately the presence of the organism on the fomite in order to prevent the spread of the organism. For example, it is known that microbes may exist in viable but nonculturable forms on fomites, or that nonculturable bacteria of selected species can be resuscitated to a culturable state under certain conditions. Often such nonculturable bacteria are present in biofilms on fomites. Accordingly, detection methods that rely on culturable forms may significantly under-report microbial contamination on fomites. The methods of the present invention, including PCR-based methods, can aid in the detection and quantification of microbial organisms, particularly nonculturable forms, by amplification and detection of cpn60-specific nucleic acid sequences.

The sample also can be a food sample. For example, the sample may be a prepared food sample, e.g., from a restaurant. Such a prepared food sample may be either cooked or raw (e.g., salads, juices). In other embodiments, the food sample may be unprocessed and/or raw, e.g., a tissue sample of an animal from a slaughterhouse, either prior to or after slaughter. The food sample may be perishable. Typically, food samples will be taken from food products such as beef, pork, poultry, seafood, dairy, fruit, vegetable, seed, nut, fungus, and grain. Dairy food samples include milk, eggs, and cheese.

Methods for collecting and storing biological and non-biological samples are generally known to those of skill in the art. For example, the Association of Analytical Communities International (AOAC International) publishes and validates sampling techniques for testing foods and agricultural products for microbial contamination. See also WO 98/32020 and U.S. Pat. No. 5,624,810 (hereby incorporated by reference in their entirety), which set forth methods and devices for collecting and concentrating microbes from the air, a liquid, or a surface. WO 98/32020, hereby incorporated by reference in its entirety, also provides methods for removing somatic cells, or animal body cells present at varying levels in certain samples.

In particular embodiments of the methods described herein, a separation and/or concentration step may be necessary to separate microbial organisms from other components of a sample or to concentrate the microbes to an amount sufficient for rapid detection. For example, a sample suspected of containing a microbial organism may require a selective enrichment of the organism (e.g., by culturing in appropriate media, e.g., for 6-96 hours or longer) prior to employing the detection methods described herein. Alternatively, appropriate filters and/or immunomagnetic separations can concentrate a microbial pathogen without the need for an extended growth stage. For example, antibodies specific for a cpn60-encoded polypeptide can be attached to magnetic beads and/or particles. Multiplexed separations, in which two or more concentration processes are employed also are contemplated, e.g., centrifugation, membrane filtration, electrophoresis, ion-exchange, affinity chromatography, and immunomagnetic separations.

Certain air or water samples may need to be concentrated. For example, certain air sampling methods require the passage of a prescribed volume of air over a filter to trap any microbial organisms, followed by isolation of the organisms into a buffer or liquid culture. Alternatively, the focused air is passed over a plate (e.g., agar) medium for growth of any microbial organisms.

Methods for sampling a tissue or a fomite with a swab are known to those of skill in the art. Generally, a swab is hydrated (e.g., with an appropriate buffer, such as Cary-Blair medium, Stuart's medium, Amie's medium, PBS, buffered glycerol saline, or water) and used to sample an appropriate surface (a fomite or tissue) for a microbial organism. Any microbe present is then recovered from the swab, such as by centrifugation of the hydrating fluid away from the swab, removal of supernatant, and resuspension of centrifugate in an appropriate buffer, or by washing of the swab with additional diluent or buffer. The recovered sample then may be analyzed according to the methods described herein for the presence of a microbial pathogen. Alternatively, the swab may be used to culture a liquid or plate (e.g., agar) medium in order to promote the growth of any pathogen for later testing. Suitable swabs include both cotton and sponge swabs; see, for example, those provided by Tecra®, such as the Tecra ENVIROSWAB®.

Samples can be processed (e.g., by nucleic acid extraction methods and/or kits known in the art) to release nucleic acid or in some cases, a biological sample can be contacted directly with PCR reaction components and appropriate oligonucleotide primers and probes.

Real-Time PCR and FRET

Nucleic acid-based methods for quantitating the amount of a microbial organism in a sample can include amplification of a cpn60 nucleic acid. Amplification methods such as PCR provide powerful means by which to increase the amount of a particular nucleic acid sequence. Nucleic acid hybridization also can be included in determining a microbial profile. Probing amplification products with species-specific hybridization probes is one of the most powerful analytical tools available for profiling. The physical matrix for hybridization can be a nylon membrane (e.g., a macroarray) or a microarray (e.g., a microchip), incorporation of one or more hybridization probes into an amplification reaction (e.g., TaqMan® or Molecular Beacon technology), solution-based methods (e.g., ORIGEN technology), or any one of numerous approaches devised for clinical diagnostics. Probes can be designed to preferentially hybridize to amplification products from individual species or to discriminate species phylogenetically. Probes designed to hybridize to nucleotide sequences from more than one species are referred to herein as “universal probes.”

U.S. Pat. Nos. 4,683,202, 4,683,195, 4,800,159, and 4,965,188 (hereby incorporated by reference in their entirety) disclose conventional PCR techniques. PCR typically employs two oligonucleotide primers that bind to a selected nucleic acid template (e.g., DNA or RNA). Primers useful in the present invention include oligonucleotide primers capable of acting as a point of initiation of nucleic acid synthesis within or adjacent to cpn60 sequences (see below). A primer can be purified from a restriction digest by conventional methods, or can be produced synthetically. Primers typically are single-stranded for maximum efficiency in amplification, but a primer can be double-stranded. Double-stranded primers are first denatured (e.g., treated with heat) to separate the strands before use in amplification. Primers can be designed to amplify a nucleotide sequence from a particular microbial species, or can be designed to amplify a sequence from more than one species. Primers that can be used to amplify a nucleotide sequence from more than one species are referred to herein as “universal primers.”

PCR assays can employ template nucleic acids such as DNA or RNA, including messenger RNA (mRNA). The template nucleic acid need not be purified; it can be a minor fraction of a complex mixture, such as a microbial nucleic acid contained in animal cells. Template DNA or RNA can be extracted from a biological or non-biological sample using routine techniques such as those described in Diagnostic Molecular Microbiology: Principles and Applications (supra). Nucleic acids can be obtained from any of a number of sources, including plasmids, bacteria, yeast, viruses, organelles, and higher organisms such as plants and animals. Standard conditions for generating a PCR product are well known in the art (see, e.g., PCR Primer: A Laboratory Manual, Dieffenbach and Dveksler (eds.), Cold Spring Harbor Laboratory Press, 1995, hereby incorporated by reference in its entirety).

Once a PCR amplification product is generated, it can be detected by, for example, hybridization using FRET technology. FRET technology (see, for example, U.S. Pat. Nos. 4,996,143, 5,565,322, 5,849,489, and 6,162,603, hereby incorporated by reference in their entirety) is based on the concept that when a donor fluorescent moiety and a corresponding acceptor fluorescent moiety are positioned within a certain distance of each other, energy transfer taking place between the two fluorescent moieties can be visualized or otherwise detected and quantitated. Two oligonucleotide probes, each containing a fluorescent moiety, can hybridize to an amplification product at particular positions determined by the complementarity of the oligonucleotide probes to the target nucleic acid sequence. Upon hybridization of the oligonucleotide probes to the amplification product at the appropriate positions, a FRET signal is generated. Hybridization temperatures and times can range from about 35° C. to about 65° C. for about 10 seconds to about 1 minute. Detection of FRET can occur in real-time, such that the increase in an amplification product after each cycle of a PCR assay is detected and, in some embodiments, quantified.

Fluorescent analysis and quantification can be carried out using, for example, a photon counting epifluorescent microscope system (containing the appropriate dichroic mirror and filters for monitoring fluorescent emission in a particular range of wavelengths), a photon counting photomultiplier system, or a fluorometer. Excitation to initiate energy transfer can be carried out with an argon ion laser, a high intensity mercury arc lamp, a fiber optic light source, or another high intensity light source appropriately filtered for excitation in the desired range.

Fluorescent moieties can be, for example, a donor moiety and a corresponding acceptor moiety. As used herein with respect to donor and corresponding acceptor fluorescent moieties, “corresponding” refers to an acceptor fluorescent moiety having an emission spectrum that overlaps the excitation spectrum of the donor fluorescent moiety. The wavelength maximum of the emission spectrum of an acceptor fluorescent moiety typically should be at least 100 nm greater than the wavelength maximum of the excitation spectrum of the donor fluorescent moiety, such that efficient non-radiative energy transfer can be produced therebetween.

Fluorescent donor and corresponding acceptor moieties are generally chosen for (a) high efficiency Förster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen with an excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC™-Red 640, LC™-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, and other chelates of Lanthanide ions (e.g., Europium, or Terbium). Donor and acceptor fluorescent moieties can be obtained from, for example, Molecular Probes, Inc. (Eugene, Ore.) or Sigma Chemical Co. (St. Louis, Mo.).

Donor and acceptor fluorescent moieties can be attached to probe oligonucleotides via linker arms. The length of each linker arm is important, as the linker arms will affect the distance between the donor and acceptor fluorescent moieties. The length of a linker arm for the purpose of the present invention is the distance in Angstroms (Å) from the nucleotide base to the fluorescent moiety. In general, a linker arm is from about 10 to about 25 Å in length. The linker arm may be of the kind described in WO 84/03285, for example. WO 84/03285 (hereby incorporated by reference in its entirety) also discloses methods for attaching linker arms to a particular nucleotide base, as well as methods for attaching fluorescent moieties to a linker arm.

An acceptor fluorescent moiety such as an LC™-Red 640-NHS-ester can be combined with C6-Phosphoramidites (available from ABI (Foster City, Calif.) or Glen Research (Sterling, Va.)) to produce, for example, LC™-Red 640-Phosphoramidite. Linkers frequently used to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as fluorescein-CPG from BioGenex (San Ramon, Calif.)), or 3′-amino-CPG's that require coupling of a fluorescein-NHS-ester after oligonucleotide synthesis.

cpn60 Nucleic Acids for Quantifying Microbial Organisms

The term “nucleic acid” as used herein encompasses both RNA and DNA, including genomic DNA. A nucleic acid can be double-stranded or single-stranded. The choice of target nucleic acid sequence to use for quantifying a microbial organism (e.g., when determining a quantitative microbial profile) depends on whether the sequences provide both broad coverage and discriminatory power. Ideally, the target should be present in all members of a given microbial community and be amplified from each member with equal efficiency using common primers, yet have distinct sequences. cpn60 (also known as hsp60 or GroEL) nucleic acid sequences are particularly useful targets for determining a microbial profile by amplification and hybridization. Chaperonin proteins are molecular chaperones required for proper folding of polypeptides in vivo. cpn60 is found universally in prokaryotes and in the organelles of eukaryotes, and can be used as a species-specific target and/or probe for identification and classification of microorganisms. Sequence diversity of this protein-encoding gene between and within bacterial genera appears greater than that of 16S rDNA sequences, making cpn60 a superior target sequence with more distinguishing power for microbial identification at the species level than 16S rDNA.

The invention provides methods to detect and quantify the amount of cpn60-containing microbial species by amplifying a portion of the cpn60 nucleic acid and/or by hybridizing to all or a portion of the cpn60 nucleic acid. cpn60 nucleic acid sequences other than those exemplified herein also can be used to detect and quantify cpn60-containing microbes in a sample and are known to those of skill in the art. Sequences of cpn60 nucleic acids from many microbes are available (see, for example, GenBank Accession Nos. NC_{—}003366, NC_{—}000913, AL939121, NC_{—}002163, and NC_{—}003198 (hereby incorporated by reference in their entirety); SEQ ID NOS:1-5, respectively). See also, U.S. Pat. No. 6,497,880 (hereby incorporated by reference in its entirety), describing the sequences of Aspergillus fumigatus cpn60 and Candida glabrata cpn60.

Also provided herein are primers and probes that can be used to amplify and detect cpn60 nucleic acid molecules. As used herein, the term “cpn60 primers” refers to oligonucleotide primers that preferentially anneal within or adjacent to cpn60 nucleic acid sequences and initiate synthesis of cpn60 nucleic acids therefrom under appropriate conditions. Primers that amplify a microbial cpn60 nucleic acid sequence (e.g., a Clostridium perfringens cpn60 sequence) can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection, similar melting temperatures for the members of a pair of primers, and the length of each primer (i.e., the primers need to be long enough to anneal with sequence-specificity and to initiate synthesis, but not so long that fidelity is reduced during oligonucleotide synthesis). Typically, oligonucleotide primers are 15 to 30 nucleotides in length (e.g., 15, 16, 17, 18, 19, 20, 21, 22, 23, 24, 25, 26, 27, 28, 29, or 30 nucleotides in length).

PCR oligonucleotide primers (SEQ ID NOS:6 and 7) that universally amplify a 552-558 base pair (bp) segment of cpn60 from numerous microorganisms have been generated (see, for example, U.S. Pat. Nos. 5,708,160 and 5,989,821, hereby incorporated by reference in their entirety), and the nucleotide sequences of the amplified cpn60 segments have been evaluated as a tool for microbial analysis. The utility of the sequence diversity in cpn60 has been demonstrated, in part, by cross hybridization experiments using nylon membranes spotted with cpn60 amplification products from typed strains probed with labeled amplification product from unknown isolates. By manipulating stringency conditions, hybridization can be limited to targets having >75% identity (e.g., >80%, >85%, >90%, >95% identify) to the unknown isolate. This level of cross hybridization allows for clear differentiation of species within genera.

Species-specific primers also can be generated. For example, primers having the sequences 5′-AAATGTAACAGCAGGGGCA-3′ (SEQ ID NO:8) and 5′-TGAAATTGCAGCAACTCTAGC-3′ (SEQ ID NO:9) can be used to specifically amplify cpn60 sequences from C. perfringens (see Example 1, below). Other examples of species-specific primers are provided in Examples 2-4. Primers specific to cpn60 sequences from other microbial organisms can readily be generated by one of ordinary skill in the art. For example, cpn60 nucleotide sequences from two or more microbial species can be aligned to identify variable regions (i.e., regions in which the nucleotide sequences vary between species), and primers can be prepared that hybridize to these regions.

As used herein, the term “cpn60 probes” refers to oligonucleotide probes that anneal preferentially to cpn60 nucleic acids, e.g., cpn60 amplification products or chromosomal cpn60 sequences. Designing oligonucleotides to be used as hybridization probes can be performed in a manner similar to the design of primers. Species-specific probes can be designed to hybridize preferentially to cpn60 nucleotide sequences from a particular microbial species. Examples of species-specific probes include those disclosed in Examples 1-4, below. Universal probes can be designed to hybridize to a target sequence that contains polymorphisms or mutations, thereby allowing for differential detection of cpn60-containing species. Such differential detection can be based either on absolute hybridization of different probes corresponding to particular species, or differential melting temperatures between, for example, a universal probe and each amplification product corresponding to a cpn60-containing species. As with oligonucleotide primers, oligonucleotide probes used in pairs typically have similar melting temperatures, and the length of each probe must be sufficient for sequence-specific hybridization to occur but not so long that fidelity is reduced during synthesis. cpn60 oligonucleotide probes used for hybridization to cpn60 amplification products generally are 15 to 30 nucleotides in length (e.g., 15, 16, 17, 18, 19, 20, 21, 22, 23, 24, 25, 26, 27, 28, 29, or 30 nucleotides in length).

Detection and Quantification of cpn60-Containing Microbial Species

Using commercially available real-time PCR instrumentation (e.g., LightCycler™, Roche Molecular Biochemicals, Indianapolis, Ind.), PCR amplification, detection, and quantification of an amplification product can be combined in a single closed cuvette with dramatically reduced cycling time. Since detection and quantification occur concurrently with amplification, real-time PCR methods obviate the need for manipulation of the amplification product, and diminish the risk of cross-contamination between amplification products. Real-time PCR greatly reduces turn-around time and is an attractive alternative to conventional PCR techniques in the clinical laboratory, in the field, or at the point of care.

Conventional PCR methods in conjunction with FRET technology can be used to practice the methods of the invention. In one embodiment, a LightCycler™ instrument is used. A detailed description of the LightCycler™ System and real-time and on-line monitoring of PCR can be found at the Roche website. The following patent applications describe real-time PCR as used in the LightCycler™ technology: WO 97/46707, WO 97/46714, and WO 97/46712, hereby incorporated by reference in their entirety. The LightCycler™ instrument is a rapid thermal cycler combined with a microvolume fluorometer utilizing high quality optics. This rapid thermocycling technique uses thin glass cuvettes as reaction vessels. Heating and cooling of the reaction chamber are controlled by alternating heated and ambient air. Due to the low mass of air and the high ratio of surface area to volume of the cuvettes, very rapid temperature exchange rates can be achieved within the LightCycler™ thermal chamber. Addition of selected fluorescent dyes to the reaction components allows the PCR to be monitored in real-time and on-line. Furthermore, the cuvettes serve as an optical element for signal collection (similar to glass fiber optics), concentrating the signal at the tip of the cuvette. The effect is efficient illumination and fluorescent monitoring of microvolume samples.

The LightCycler™ carousel that houses the cuvettes can be removed from the instrument. Therefore, samples can be loaded outside of the instrument (in a PCR Clean Room, for example). In addition, this feature allows for the sample carousel to be easily cleaned and sterilized. The fluorometer, as part of the LightCycler™ apparatus, houses the light source. The emitted light is filtered and focused by an epi-illumination lens onto the top of the cuvette. Fluorescent light emitted from the sample is then focused by the same lens, passed through a dichroic mirror, filtered appropriately, and focused onto data-collecting photohybrids. The optical unit currently available in the LightCycler™ instrument (Roche Molecular Biochemicals, Catalog No. 2 011 468) includes three band-pass filters (530 nm, 640 nm, and 710 nm), providing three-color detection and several fluorescence acquisition options. Data collection options include once per cycling step monitoring, fully continuous single-sample acquisition for melting curve analysis, continuous sampling (in which sampling frequency is dependent on sample number) and/or stepwise measurement of all samples after defined temperature interval.

The LightCycler™ can be operated and the data retrieved using a PC workstation and a Windows operating system. Signals from the samples are obtained as the machine positions the capillaries sequentially over the optical unit. The software can display the presence and amount of fluorescent signals in real-time immediately after each measurement. Fluorescent acquisition time is 10-100 milliseconds (msec). After each cycling step, a quantitative display of fluorescence vs. cycle number can be continually updated for all samples. The generated data can be stored for further analysis.

Real-time PCR methods include multiple cycling steps, each step including an amplification step and a hybridization step. In addition, each cycling step typically is followed by a FRET detecting step to detect hybridization of one or more probes to an amplification product. The presence of an amplification product is indicative of the presence of one or more cpn60-containing species. As used herein, “cpn60-containing species” refers to microbial species that contain cpn60 nucleic acid sequences. Generally, the presence of FRET indicates the presence of one or more cpn60-containing species in the sample, and the absence of FRET indicates the absence of a cpn60-containing species in the sample. Typically, detection of FRET within, for example, 20, 25, 30, 35, 40, or 45 cycling steps is indicative of the presence of a cpn60-containing species.

As described herein, cpn60 amplification products can be detected using labeled hybridization probes that take advantage of FRET technology. A common format of FRET technology utilizes two hybridization probes that generally are designed to hybridize in close proximity to each other, where one probe is labeled with a donor fluorescent moiety and the other is labeled with a corresponding acceptor fluorescent moiety. Thus, two cpn60 probes can be used, one labeled with a donor fluorophore and the other labeled with a corresponding acceptor fluorophore. The presence of FRET between the donor fluorescent moiety of the first cpn60 probe and the corresponding acceptor fluorescent moiety of the second cpn60 probe is detected upon hybridization of the cpn60 probes to the cpn60 amplification product. For example, a donor fluorescent moiety such as fluorescein is excited at 470 nm by the light source of the LightCycler™ Instrument. During FRET, the fluorescein transfers its energy to an acceptor fluorescent moiety such as LightCycler™-Red 640 (LC™-Red 640) or LightCycler™-Red 705 (LC™-Red 705). The acceptor fluorescent moiety then emits light of a longer wavelength, which is detected by the optical detection system of the LightCycler™ instrument. Efficient FRET can only take place when the fluorescent moieties are in direct local proximity and when the emission spectrum of the donor fluorescent moiety overlaps with the absorption spectrum of the acceptor fluorescent moiety. The intensity of the emitted signal can be correlated with the number of original target DNA molecules (e.g., the number of copies of cpn60).

Another FRET format can include the use of TaqMan® technology to detect the presence or absence of a cpn60 amplification product, and hence, the presence or absence of cpn60-containing species. TaqMan® technology utilizes one single-stranded hybridization probe labeled with two fluorescent moieties. When a first fluorescent moiety is excited with light of a suitable wavelength, the absorbed energy is transferred to a second fluorescent moiety according to the principles of FRET. The second fluorescent moiety generally is a quencher molecule. During the annealing step of the PCR reaction, the labeled hybridization probe binds to the target DNA (i.e., the cpn60 amplification product) and is degraded by the 5′ to 3′ exonuclease activity of the Taq Polymerase during the subsequent elongation phase. As a result, the excited fluorescent moiety and the quencher moiety become spatially separated from one another. As a consequence of excitation of the first fluorescent moiety in the absence of the quencher, the fluorescence emission from the first fluorescent moiety can be detected. By way of example, an ABI PRISM® 7700 Sequence Detection System (Applied Biosystems, Foster City, Calif.) uses TaqMan® technology, and is suitable for performing the methods described herein for detecting cpn60-containing species. Information on PCR amplification and detection using an ABI PRISM® 770 system can be found at the Applied Biosystems website (world wide web at appliedbiosystems.com/products).

Molecular beacons in conjunction with FRET also can be used to detect the presence of a cpn60 amplification product using the real-time PCR methods of the invention. Molecular beacon technology uses a hybridization probe labeled with a first fluorescent moiety and a second fluorescent moiety. The second fluorescent moiety generally is a quencher, and the fluorescent labels typically are located at each end of the probe. Molecular beacon technology uses an oligonucleotide probe having sequences that permit secondary structure formation (e.g., a hairpin). As a result of secondary structure formation within the probe, both fluorescent moieties are in spatial proximity when the probe is in solution. After hybridization to the target cpn60 amplification product, the secondary structure of the probe is disrupted and the fluorescent moieties become separated from one another such that after excitation with light of a suitable wavelength, the emission of the first fluorescent moiety can be detected.

The amount of FRET corresponds to the amount of amplification product, which in turn corresponds to the amount of template nucleic acid present in the sample. Similarly, the amount of template nucleic acid corresponds to the amount of microbial organism present in the sample. Therefore, the amount of FRET produced when amplifying nucleic acid obtained from a biological sample can be correlated to the amount of a microorganism. Typically, the amount of a microorganism in a sample can be quantified by comparing the amount of FRET produced from amplified nucleic acid obtained from known amounts of the microorganism. Accurate quantitation requires measuring the amount of FRET while amplification is increasing linearly. In addition, there must be an excess of probe in the reaction. Furthermore, the amount of FRET produced in the known samples used for comparison purposes can be standardized for particular reaction conditions, such that it is not necessary to isolate and amplify samples from every microorganism for comparison purposes.

As an alternative to FRET, a cpn60 amplification product can be detected using, for example, a fluorescent DNA binding dye (e.g., SYBRGreenI® or SYBRGold® (Molecular Probes)). Upon interaction with an amplification product, such DNA binding dyes emit a fluorescent signal after excitation with light at a suitable wavelength. A double-stranded DNA binding dye such as a nucleic acid intercalating dye also can be used. When double-stranded DNA binding dyes are used, a melting curve analysis usually is performed for confirmation of the presence of the amplification product.

Melting curve analysis is an additional step that can be included in a cycling profile. Melting curve analysis is based on the fact that a nucleic acid sequence melts at a characteristic temperature (Tm), which is defined as the temperature at which half of the DNA duplexes have separated into single strands. The melting temperature of a DNA molecule depends primarily upon its nucleotide composition. A DNA molecule rich in G and C nucleotides has a higher Tm than one having an abundance of A and T nucleotides. The temperature at which the FRET signal is lost correlates with the melting temperature of a probe from an amplification product. Similarly, the temperature at which signal is generated correlates with the annealing temperature of a probe with an amplification product. The melting temperature(s) of cpn60 probes from an amplification product can confirm the presence or absence of cpn60-containing species in a sample, and can be used to quantify the amount of a particular cpn60-containing species. For example, a universal probe that hybridizes to a variable region within cpn60 will have a Tm that depends upon the sequence to which it hybridizes. Thus, a universal probe may have a Tm of 70° C. when hybridized to a cpn60 amplification product generated from one microbial organism, but a Tm of 65° C. when hybridized to a cpn60 amplification product generated from a second microbial organism. By observing a temperature-dependent, step-wise decrease in fluorescence of a sample as it is heated, the particular cpn60-containing species in the sample can be identified and the relative amounts of the species in the sample can be determined.

Within each thermocycler run, control samples can be cycled as well. Positive control samples can amplify a nucleic acid control template (e.g., a nucleic acid other than cpn60) using, for example, control primers and control probes. Positive control samples also can amplify, for example, a plasmid construct containing a cpn60 nucleic acid molecule. Such a plasmid control can be amplified internally (e.g., within the sample) or in a separate sample run side-by-side with the test samples. Each thermocycler run also should include a negative control that, for example, lacks cpn60 template DNA. Such controls are indicators of the success or failure of the amplification, hybridization and/or FRET reaction. Therefore, control reactions can readily determine, for example, the ability of primers to anneal with sequence-specificity and to initiate elongation, as well as the ability of probes to hybridize with sequence-specificity and for FRET to occur.

In one embodiment, methods of the invention include steps to avoid contamination. For example, an enzymatic method utilizing uracil-DNA glycosylase is described in U.S. Pat. Nos. 5,035,996, 5,683,896 and 5,945,313 (hereby incorporated by reference in their entirety), and can be used to reduce or eliminate contamination between one thermocycler run and the next. In addition, standard laboratory containment practices and procedures are desirable when performing methods of the invention. Containment practices and procedures include, but are not limited to, separate work areas for different steps of a method, containment hoods, barrier filter pipette tips and dedicated air displacement pipettes. Consistent containment practices and procedures by personnel are necessary for accuracy in a diagnostic laboratory handling clinical samples.

It is understood that the present invention is not limited by the configuration of one or more commercially available instruments.

Articles of Manufacture

The invention also provides articles of manufacture. Articles of manufacture can include at least one cpn60 oligonucleotide primer, as well as instructions for using the cpn60 oligonucleotide(s) to quantify the amount of one or more microbial organisms in a biological or non-biological sample.

In one embodiment, the cpn60 oligonucleotide(s) are attached to a microarray (e.g., a GeneChip®, Affymetrix, Santa Clara, Calif.). In another embodiment, an article of manufacture can include one or more cpn60 oligonucleotide primers and one or more cpn60 oligonucleotide probes. Such cpn60 primers and probes can be used, for example, in real-time time amplification reactions to amplify and simultaneously detect cpn60 amplification products.

Suitable oligonucleotide primers include those that are complementary to highly conserved regions of cpn60 and that flank a variable region. Such universal cpn60 primers can be used to specifically amplify these variable regions, thereby providing a sequence with which to identify microorganisms. Examples of cpn60 oligonucleotide primers include the following:

| | |
|---|---|
| 5′-GAIIIIGCIGGIGA(T/C)GGIACIACIAC-3′ | (SEQ ID NO:6) |
| 5′-(T/C)(T/G)I(T/C)(T/G)ITCICC(A/G)AAICCIGGIGC(T/C)TT-3′ | (SEQ ID NO:7) |

Suitable oligonucleotide primers also include those that are complementary to species-specific cpn60 sequences, and thus result in an amplification product only if a particular species is present in the sample. Examples of species-specific primers include the following:

| | |
|---|---|
| 5′-AAATGTAACAGCAGGGGCA-3′ | (SEQ ID NO:8) |
| 5′-TGAAATTGCAGCAACTCTAGC-3′ | (SEQ ID NO:9) |
| 5′-GTCCATCATTACCGAAGGCT-3′ | (SEQ ID NO:10) |
| 5′-ATCGCTTTAGAGTCGGAGCA-3′ | (SEQ ID NO:11) |
| 5′-AAAATGACAGTAGAGCTTTCAAGC-3′ | (SEQ ID NO:12) |
| 5′-TTATTTACAACCAAAGTTGCAAGC-3′ | (SEQ ID NO:13) |
| 5′-GGCTATCATCACTGAAGGTCTG-3′ | (SEQ ID NO:14) |
| 5′-TTCTTCAACTGCAGCGGTAAC-3′ | (SEQ ID NO:15) |

Similar to cpn60 oligonucleotide primers, cpn60 oligonucleotide probes generally are complementary to cpn60 sequences. cpn60 oligonucleotide probes can be designed to hybridize universally to cpn60 sequences, or can be designed for species-specific hybridization to the variable region of cpn60 sequences. Examples of useful species-specific cpn60 probes include the following:

| | |
|---|---|
| 5′-ATGTCTTCTTTTCCATTTACAGGCTTAGAA-3′ | (SEQ ID NO:16) |
| 5′-TACGGACAGGGCTTTCAGCTCTTCA-3′ | (SEQ ID NO:17) |
| 5′-CTTCACCTTCAATATCTTCAGCGATAATTAAAAGT-3′ | (SEQ ID NO:18) |
| 5′-TGTTGCTGCGGGCATGAACC-3′ | (SEQ ID NO:19) |

An article of manufacture of the invention can further include additional components for carrying out amplification reactions and/or reactions, for example, on a microarray. Articles of manufacture for use with PCR reactions can include nucleotide triphosphates, an appropriate buffer, and a polymerase. An article of manufacture of the invention also can include appropriate reagents for detecting amplification products. For example, an article of manufacture can include one or more restriction enzymes for distinguishing amplification products from different species of microorganism, or can include fluorophore-labeled oligonucleotide probes for real-time detection of amplification products.

It will be appreciated by those of ordinary skill in the art that different articles of manufacture can be provided to evaluate microbial profiles of different types of samples (e.g., biological samples from different types of animals). For example, the microbial profile of the pig GIT has a different community of microbes than that of poultry. Therefore, an article of manufacture designed to evaluate the microbial profile of, for example, the pig GIT may have a different set of controls or a different set of species-specific hybridization probes than that designed for poultry. Alternatively, a more generalized article of manufacture can be used to evaluate the microbial profiles of a number of different animal species.

The invention will be further described in the following examples, which do not limit the scope of the invention described in the claims.

### EXAMPLES

### Example 1

### Specific Real-Time PCR Detection of cpn60 from Clostridium perfringens

DNA was extracted from C. perfringens, C. speticum, C. chauvoei, C. difficile, Escherichia coli, Campylobacter jejuni, Salmonella enterica, Lactobacillus fermentus, Bifidobacterium animalis, Mycobacterium avium, and ileal contents of pigs fed corn, wheat or barley. Real-time PCR was conducted using 500 nM forward primer CperfCPN41U19 (5′-AAATGTAACAGCAGGGGCA-3′; SEQ ID NO:8), 500 nM reverse primer CperfCPN160L21 (5′-TGAAATTGCAGCAACTCTAGC-3′; SEQ ID NO:9), 200 nM Taqman probe CperfCPN60-probe129L30 (5′-ATGTCTTCTTTTCCATTTACAGGCTTAGAA-3′; SEQ ID NO:16), and 1 μL DNA template in a total reaction volume of 25 μL. PCR conditions included one Predwell cycle of 2 minutes at 95° C. and 2 minutes at 50° C., followed by 40 cycles of annealing for 30 seconds at 59° C. and denaturation for 30 seconds at 94° C. Reactions were run in triplicate, and control samples did not contain template DNA.

Relative fluorescence units (RFU) were measured after subtraction of baseline fluorescence activity during real-time PCR. Only amplification in the presence of template DNA from C. perfringens resulted in significant quantities of the expected 139 bp product. Above baseline fluorescence was observed with the C. perfringens template at calculated threshold cycles of 27.4, 27.6, and 27.6 for the three replicates.

To assess the sensitivity of the assay, real-time PCR was conducted in triplicate with no template DNA and with a 10-fold dilution series of template DNA from C. perfringens, and the number of PCR cycles required for fluorescence above baseline was calculated. This value is referred to as Threshold Cycle (C_{T}). Results are shown in Table 1. To further assess sensitivity, real-time PCR also was run with a 2-fold dilution series of template DNA from C. perfringens. Results are shown in Table 2.

TABLE 1

| Dilution of DNA template | C_{T} | Mean C_{T} |
|---|---|---|
| 1:10 | 19.0, 19.2, 18.9 | 19.0 |
| 1:100 | 20.9, 20.7, 20.7 | 20.8 |
| 1:1,000 | 23.7, 23.8, 23.6 | 23.7 |
| 1:10,000 | 27.3, 27.3, 27.5 | 27.4 |
| 1:100,000 | 30.5, 30.6, 30.3 | 30.5 |
| 1:1,000,000 | 35.9, 34.3, 34.2 | 34.8 |
| No template | — | — |

TABLE 2

| Dilution of DNA template | C_{T} | Mean C_{T} |
|---|---|---|
| 1:100 | 23.7, 24.0, 24.1 | 23.9 |
| 1:200 | 24.9, 24.7, 25.1 | 24.9 |
| 1:400 | 25.6, 25.4, 25.7 | 25.6 |
| 1:800 | 27.3, 27.0, 27.3 | 27.2 |
| 1:1,600 | 27.7, 27.7, 27.9 | 27.8 |
| 1:3,200 | 28.7, 28.8, 28.7 | 28.7 |
| No template | — | — |

### Example 2

### Specific Real-Time PCR Detection of cpn60 from Salmonella spp.

DNA was extracted from Salmonella enterica, Clostridium perfringens, C. speticum, C. chauvoei, C. difficile, Escherichia coli, Campylobacter jejuni, Lactobacillus fermentus, Bifidobacterium animalis, Mycobacterium avium, and ileal contents of pigs fed corn, wheat or barley. Real-time PCR was conducted using 500 nM forward primer Salmone1CPN18U20 (5′-GTCCATCATTACCGAAGGCT-3′; SEQ ID NO:10), 500 nM reverse primer Salmone1CPN139L20 (5′-ATCGCTTTAGAGTCGGAGCA-3′; SEQ ID NO:11), 200 nM Taqman probe Salmone1Probe 111L25 (5′-TACGGACAGGGCTTTCAGCTCTTCA-3′; SEQ ID NO:17), and 1 μL DNA template in a total reaction volume of 25 μL. PCR conditions included one Predwell cycle of 2 minutes at 95° C. and 2 minutes at 50° C., followed by 40 cycles of annealing for 30 seconds at 60° C. and denaturation for 30 seconds at 94° C. Reactions were run in triplicate, and control samples did not contain template DNA.

RFU were measured after subtraction of baseline fluorescence activity during real-time PCR. Only amplification in the presence of template DNA from S. enterica or from the ileal contents of corn-fed pigs resulted in significant quantities of the expected 141 bp product. Above baseline fluorescence was observed with the S. enteritidis template at calculated C_{T} of 21.6, 23.5, and 20.5 for the three replicates.

To assess sensitivity, real-time PCR was conducted in triplicate with no template DNA and with a 10-fold dilution series of template DNA from S. enteriditis. Results are shown in Table 3. S. enteriditis cell counts were 7.75×10⁸ cells/mL using a Petroff-Houser cell counter (i.e., total live and dead cells). The total viable cell count was 3.8×10⁶ cfu/mL. The equivalent of 1 mL cell culture media was extracted and resuspended in 100 μL buffer. Values in the first column of Table 3 indicate the number of cells represented in 1 μL of extracted DNA used in the real-time PCR assay. Thus, this assay was able to detect cpn60 in a DNA sample from as few as 77.5 cells.

TABLE 3

| # extracted S. enteritidis | C_{T} | Mean C_{T} |
|---|---|---|
| 7.75 × 10⁵ | 24.2, 25.4, 25.6 | 25.1 |
| 7.75 × 10⁴ | 26.9, 26.6, 27.0 | 26.8 |
| 7.75 × 10³ | 30.0, 30.4, 30.0 | 30.1 |
| 7.75 × 10² | 33.7, 34.1, 33.8 | 33.9 |
| 7.75 × 10¹ | 36.9, 37.2, 36.3 | 36.8 |
| 7.75 | — | — |
| No template | — | — |

### Example 3

### Specific Real-Time PCR Detection of cpn60 from Campylobacter jejuni

DNA was extracted from Campylobacter jejuni, Campylobacter coli, Salmonella enterica, Clostridium perfringens, C. speticum, C chauvoei, Escherichia coli, Lactobacillus fermentus, Mycobacterium avium, and ileal contents of pigs fed corn, wheat or barley. Real-time PCR was conducted using 500 nM forward primer CampJejCPN355U24 (5′-AAAATGACAGTAGAGCTTTCAAGC-3′; SEQ ID NO:12), 500 nM reverse primer CampJejCPN501L24 (5′-TTATTTACAACCAAAGTTGCAAGC-3′; SEQ ID NO:13), 200 nM Taqman probe CampJejCPN60 probe465L35 (5′-CTTCACCTTCAATATCTTCAGCGATAATTAAAAGT-3′; SEQ ID NO:18), and 1 μL DNA template in a total reaction volume of 25 μL. PCR conditions included one Predwell cycle of 2 minutes at 95° C. and 2 minutes at 50° C., followed by 40 cycles of annealing for 30 seconds at 55° C. and denaturation for 30 seconds at 94° C. Reactions were run in triplicate, and control samples did not contain template DNA.

RFU were measured after subtraction of baseline fluorescence activity during real-time PCR. Only amplification in the presence of template DNA from C. jejuni resulted in significant quantities of the expected 170 bp product. To quantify the amount of C. jejuni DNA, real-time PCR was conducted in duplicate with no template DNA and with a 2-fold dilution series of template DNA from C. jejuni. Above baseline fluorescence was observed with the C. jejuni template at calculated C_{T} of 31.1, 32.4, and 33.1 for each duplicate in the 2-fold dilution series.

### Example 4

### Specific Real-Time PCR Detection of cpn60 from Escherichia coli/Shiaella spp.

DNA was extracted from Escherichia coli, Shigella boydii, Clostridium perfringens, C. difficile, C. chauvoei, Campylobacter jejuni, Salmonella enterica, Lactobacillus fermentus, Bifidobacterium animalis, Mycobacterium avium, and ileal contents of pigs fed corn, wheat or barley. Real-time PCR was conducted using 500 nM forward primer Ecoli-shigCPN18U22 (5′-GGCTATCATCACTGAAGGTCTG-3′; SEQ ID NO:14), 500 nM reverse primer Ecoli-shigCPN117L21 (5′-TTCTTCAACTGCAGCGGTAAC-3′; SEQ ID NO:15), 200 nM Taqman probe Ecoli-shig-probe-CPN48U20 (5′-TGTTGCTGCGGGCATGAACC-3′; SEQ ID NO:19), and 1 μL DNA template in a total reaction volume of 25 μL. PCR conditions included one Predwell cycle of 2 minutes at 95° C. and 2 minutes at 50° C., followed by 40 cycles of annealing for 30 seconds at 61° C. and denaturation for 30 seconds at 94° C. Reactions were run in triplicate, and control samples did not contain template DNA.

RFU were measured after subtraction of baseline fluorescence activity during real-time PCR. Amplification in the presence of template DNA from E. coli and S. Boydii resulted in significant quantities of the expected 100 bp product (see Table 4). Lesser amounts of the expected product also were observed with template DNA from ileal contents of pigs and from B. animalis.

TABLE 4

| DNA template | C_{T} | Mean C_{T} |
|---|---|---|
| E. coli | 17.4, 17.5, 17.7 | 17.5 |
| S. boydii | 21.6, 21.9, 21.9 | 21.8 |
| S. enterica | — | — |
| B. animalis | 29.7, 29.9, 29.6 | 29.7 |
| Ileal contents, corn-fed | 27.6, 27.9, 27.8 | 27.8 |
| Ileal contents, wheat-fed | 28.1, 28.3, 28.1 | 28.2 |
| Ileal contents, barley-fed | 29.5, 29.9, 29.6 | 29.7 |
| No template | 29.5, 30.5, — | 30.0 (n = 2) |

To assess sensitivity, real-time PCR was conducted in triplicate with no template DNA and with a 10-fold dilution series of template DNA from E. coli/Shigella spp. Results are shown in Table 5. E. coli cell counts were 8.5×10⁸ cells/mL using a Petroff-Houser cell counter. The total viable cell count was 5.8×10⁸ cfu/mL. The equivalent of 1 mL cell culture media was extracted and resuspended in 100 μL buffer. Values in the first column of Table 5 indicate the number of cells represented in 1 μL of extracted DNA used in the real-time PCR assay. Thus, cpn60 was detected from as few as 850 cells, although the background was high.

TABLE 5

| # extracted E. coli | C_{T} | Mean C_{T} |
|---|---|---|
| 8.5 × 10⁶ | 16.5, 16.6, 16.7 | 16.6 |
| 8.5 × 10⁵ | 18.8, 18.9, 18.8 | 18.8 |
| 8.5 × 10⁴ | 22.1, 22.4, 22.2 | 22.2 |
| 8.5 × 10³ | 25.5, 25.7, 25.6 | 25.6 |
| 8.5 × 10² | 28.5, 28.6, 28.1 | 28.4 |
| No template | 29.5, 29.5, 29.5 | 29.5 |

### Example 5

### Quantitating Microbial Organisms Using Universal Primers and a Universal Probe

A biological sample is obtained from poultry GIT and genomic DNA is extracted using standard methods (Diagnostic Molecular Microbiology: Principles and Applications (supra)). Real-time PCR is conducted using universal cpn60 primers having the nucleotide sequences set forth in SEQ ID NO:6 and SEQ ID NO:7, and a universal cpn60 probe having the sequence 5′-GACAAAGTCGGTAAAGAAGGCGTTATCA-3′ (SEQ ID NO:8), labeled at the 5′ end with fluorescein (fluorophore; Molecular Probes, Inc.) and at the 3′ end with dabcyl (quencher; (4-(4′-dimethylaminophenylazo)benzoic acid) succinimidyl ester; Molecular Probes, Inc.). This probe binds to a variable region of the cpn60 gene from numerous microbial species; thus the Tm of the probe from an amplification product varies depending upon the nucleotide sequence within the amplification product to which the probe hybridizes.

The PCR reaction contains 3 μL extracted DNA, 1 μM each universal cpn60 primer, 340 nM universal cpn60 probe, 2.5 units Amplitaq Gold DNA polymerase (Applied Biosystems), 0.25 mM each deoxyribonucleotide, 3.5 mM MgCl₂, 50 mM KCl, and 10 mM Tris-HCl, pH 8.0 in a total reaction volume of 50 μL. PCR conditions include an initial incubation at 95° C. for 10 minutes to activate the Amplitaq Gold DNA polymerase, followed by 40 cycles of 30 seconds at 95° C., 60 seconds at 50° C., and 30 seconds at 72° C. Fluorescence is monitored during the 50° C. annealing steps throughout the 40 cycles. After the cycling steps are complete, the melting temperature of the universal probe from the amplification products is analyzed. As the temperature is increased, the universal probe is released from the amplification product from each species' cpn60 sequence at a specific temperature, corresponding to the Tm of the universal probe and the cpn60 sequence of the particular species. The step-wise loss of fluorescence at particular temperatures is used to identify the particular species present, and the loss in fluorescence of each step compared to the total amount of fluorescence correlates with the relative amount of each microorganism.

### Example 6

### Quantification of Microbial Organisms Using Universal Primers and Species-Specific Probes

A biological sample is obtained from poultry GIT and genomic DNA is extracted using standard methods (Diagnostic Molecular Microbiology: Principles and Applications (supra)). Real-time PCR is conducted using universal cpn60 primers having the nucleotide sequences set forth in SEQ ID NO:6 and SEQ ID NO:7, and species-specific probes having the nucleotide sequences 5′-AGCCGTTGCAAAAGCAGGCAAACCGC-3′ (SEQ ID NO:9), 5′-TTGAGCAAATAGTTCAAGCAGGTAA-3′ (SEQ ID NO:10), 5′-GCAACTCTGGTTGTTAACACCATGC-3′ (SEQ ID NO:11), 5′-TGGAGAAGGTCATCCAGGCCAACGC-3′ (SEQ ID NO:12), and 5′-TAGAACAAATTCAAAAAACAGGCAA-3′ (SEQ ID NO:13). These species-specific probes hybridize to cpn60 nucleotide sequences from S. enterica, C. perfringens, E. coli, S. coelicolor, and C. jejuni, respectively. The sequences of the probes are identified by aligning cpn60 cDNA sequences from the five organisms and identifying a sequence that is unique to each particular organism (i.e., a sequence not found in the other organisms). Each of the species-specific probes is labeled with a different fluorescent moiety to allow differential detection of the various species.

The PCR reaction contains 3 μL extracted DNA, 1 μM each universal cpn60 primer, 340 nM universal cpn60 probe, 2.5 units Amplitaq Gold DNA polymerase (Applied Biosystems), 0.25 mM each deoxyribonucleotide, 3.5 mM MgCl₂, 50 mM KCl, and 10 mM Tris-HCl, pH 8.0 in a total reaction volume of 50 μL. PCR conditions include an initial incubation at 95° C. for 10 minutes to activate the Amplitaq Gold DNA polymerase, followed by 40 cycles of 30 seconds at 95° C., 60 seconds at 50° C., and 30 seconds at 72° C. Fluorescence is monitored during the 50° C. annealing steps throughout the 40 cycles, at wavelengths corresponding to the particular moieties on the probes. The amount of fluorescence detected at each of the monitored wavelengths correlates with the amount of each cpn60 amplification product. The amount of each species-specific amplification product is then correlated with the amount of each species of microbe by comparison to the amount of amplification product generated from positive control samples containing nucleic acid isolated from known amounts of each microbial species. Nucleic acids in the positive control samples can be obtained from, for example, E. coli or Salmonella spp.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for quantifying the amount of one or more microbial species in a biological or non-biological sample, said method comprising:
(a) providing said sample;
(b) subjecting said sample to amplification in the presence of cpn60 primers, thereby generating an amplification product if a microbial species containing cpn60 is present in said sample; and
(c) quantifying said amplification product,
wherein said amount of said product is correlated with the amount of said microbial species in said sample.

2. The method of claim 1, wherein said primers are universal cpn60 primers, and wherein said quantifying comprises hybridization of one or more species-specific cpn60 probes to said amplification product.

3. The method of claim 2, wherein said hybridization is detected in real time.

4. The method of claim 2, wherein said correlation employs a standard curve of known amounts of said microbial species.

5. The method of claim 2, wherein said one or more species-specific cpn60 probes are differentially labeled.

6. The method of claim 1, wherein said primers are universal cpn60 primers, and wherein said quantifying comprises hybridization of a universal cpn60 probe to said amplification product.

7. The method of claim 1, wherein said quantifying comprises hybridization of a first cpn60 probe and a second cpn60 probe to said amplification product.

8. The method of claim 7, wherein said first cpn60 probe is labeled with a donor fluorescent moiety, wherein said second cpn60 probe is labeled with a corresponding acceptor fluorescent moiety, and wherein said first and second cpn60 probes hybridize to said amplification product in a manner such that fluorescence resonance energy transfer occurs.

9. The method of claim 8, wherein said donor fluorescent moiety is fluorescein.

10. The method of claim 8, wherein said corresponding acceptor fluorescent moiety is selected from the group consisting of LC-Red 640, LC-Red 705, Cy5, and Cy5.5.

11. The method of claim 1, wherein said quantifying comprises hybridization of one cpn60 probe to said amplification product.

12. The method of claim 11, wherein said cpn60 probe is labeled with a donor fluorescent moiety and a corresponding acceptor fluorescent moiety.

13. The method of claim 12, wherein said cpn60 probe comprises a nucleotide sequence that permits secondary structure formation, wherein said secondary structure formation results in spatial proximity between said first and second fluorescent moieties.

14. The method of claim 1, wherein said quantifying comprises measuring the interaction of a fluorescent dye with said amplification product.

15. The method of claim 14, wherein said interaction is intercalation.

16. The method of claim 1, wherein said sample is selected from the group consisting of a biological tissue, a biological fluid, a biological elimination product, a water sample, a soil sample, and a swab from an inanimate object.

17. The method of claim 1, wherein said one or more microbial species belong to genera selected from the group consisting of Escherichia, Salmonella, Campylobacter, Staphyococcus, Clostridium, Pseudomonas, Bifidobacterium, Bacillus, Enterococcus, Acanthamoeba, Cryptosporidium, Tetrahymena, Aspergillus, Candida, and Saccharomyces.

18. An article of manufacture comprising one or more cpn60 primers and one or more cpn60 probes, and instructions for using said one or more cpn60 primers and one or more cpn60 probes for quantifying the amount of one or more microbial species in a biological or non-biological sample.

19. A method for quantifying the amount of a particular microbial species in a biological or environmental sample, said method comprising:
(a) providing said sample;
(b) subjecting said sample to amplification in the presence of primers specific to the cpn60 gene of said microbial species, thereby generating an amplification product if said microbial species is present in said sample; and
(c) quantifying said amplification product,
wherein said amount of said product is correlated with the amount of said microbial species in said sample.

20. The method of claim 19, wherein said quantifying comprises hybridization of a first cpn60 probe and a second cpn60 probe to said amplification product.

21. The method of claim 20, wherein said first cpn60 probe is labeled with a donor fluorescent moiety, wherein said second cpn60 probe is labeled with a corresponding acceptor fluorescent moiety, and wherein said first and second cpn60 probes hybridize to said amplification product in a manner such that fluorescence resonance energy transfer occurs.

22. The method of claim 21, wherein said donor fluorescent moiety is fluorescein.

23. The method of claim 21, wherein said corresponding acceptor fluorescent moiety is selected from the group consisting of LC-Red 640, LC-Red 705, Cy5, and Cy5.5.

24. The method of claim 19, wherein said quantifying comprises hybridization of one cpn60 probe to said amplification product.

25. The method of claim 24, wherein said cpn60 probe is labeled with a donor fluorescent moiety and a corresponding acceptor fluorescent moiety.

26. The method of claim 24, wherein said cpn60 probe comprises a nucleotide sequence that permits secondary structure formation, wherein said secondary structure formation results in spatial proximity between said first and second fluorescent moieties.

27. The method of claim 19, wherein said quantifying comprises interaction of a fluorescent dye with said amplification product.

28. The method of claim 27, wherein said interaction is intercalation.

29. The method of claim 19, wherein said sample is selected from the group consisting of a biological tissue, a biological fluid, a biological elimination product, a water sample, a soil sample, and a swab from an inanimate object.

30. The method of claim 19, wherein said microbial species belongs to a genera selected from the group consisting of Escherichia, Salmonella, Campylobacter, Staphyococcus, Clostridium, Pseudomonas, Bifidobacterium, Bacillus, Enterococcus, Acanthamoeba, Cryptosporidium, Tetrahymena, Aspergillus, Candida, and Saccharomyces.

31. A method for quantifying the amount of Clostridium perfringens in a biological or non-biological sample, the method comprising:
(a) providing said sample;
(b) subjecting said sample to amplification in the presence of cpn60 primers, thereby generating an amplification product if said C. perfringens is present in said sample; and
(c) quantifying said amplification product by hybridizing a cpn60 probe to said product,
wherein said amount of said amplification product is correlated with the amount of said C. perfringens in said sample.

32. The method of claim 31, wherein said cpn60 primers have the nucleotide sequences set forth in SEQ ID NO:8 and SEQ ID NO:9.

33. The method of claim 31, wherein said cpn60 probe has the nucleotide sequence set forth in SEQ ID NO:16.

34. A method for quantifying the amount of Salmonella enterica in a biological or non-biological sample, said method comprising:
(a) providing said sample;
(b) subjecting said sample to amplification in the presence of cpn60 primers, thereby generating an amplification product if said S. enterica is present in said sample; and
(c) quantifying said amplification product by hybridizing a cpn60 probe to said product,
wherein said amount of said amplification product is correlated with the amount of said S. enterica in said sample.

35. The method of claim 34, wherein said cpn60 primers have the nucleotide sequences set forth in SEQ ID NO:10 and SEQ ID NO:11.

36. 33. The method of claim 34, wherein said cpn60 probe has the nucleotide sequence set forth in SEQ ID NO:17.

37. A method for quantifying the amount of Campylobacter jejuni in a biological or non-biological sample, said method comprising:
(a) providing said sample;
(b) subjecting said sample to amplification in the presence of cpn60 primers, thereby generating an amplification product if said C. jejuni is present in said sample; and
(c) quantifying said amplification product by hybridizing a cpn60 probe to said product,
wherein said amount of said amplification product is correlated with the amount of said C. jejuni in said sample.

38. The method of claim 37, wherein said cpn60 primers have the nucleotide sequences set forth in SEQ ID NO:12 and SEQ ID NO:13.

39. The method of claim 37, wherein said cpn60 probe has the nucleotide sequence set forth in SEQ ID NO:18.

40. A method for quantifying the amount of Escherichia coli in a biological or non-biological sample, said method comprising:
(a) providing said sample;
(b) subjecting said sample to amplification in the presence of cpn60 primers, thereby generating an amplification product if said E. coli is present in said sample; and
(c) quantifying said amplification product by hybridizing a cpn60 probe to said product,
wherein said amount of said amplification product is correlated with the amount of said E. coli in said sample.

41. The method of claim 40, wherein said cpn60 primers have the nucleotide sequences set forth in SEQ ID NO:14 and SEQ ID NO:15.

42. The method of claim 40, wherein said cpn60 probe has the nucleotide sequence set forth in SEQ ID NO:19.

43. An article of manufacture comprising cpn60 primers having the nucleotide sequences set forth in SEQ ID NO:8 and SEQ ID NO:9, and a cpn60 probe having the nucleotide sequence set forth in SEQ ID NO:16.

44. The article of manufacture of claim 43, further comprising instructions for using said cpn60 primers and probes to quantify the amount of C. perfringens in a biological or non-biological sample.

45. An article of manufacture comprising cpn60 primers having the nucleotide sequences set forth in SEQ ID NO:10 and SEQ ID NO:11, and a cpn60 probe having the nucleotide sequence set forth in SEQ ID NO:17.

46. The article of manufacture of claim 45, further comprising instructions for using said cpn60 primers and probes to quantify the amount of S. enterica in a biological or non-biological sample.

47. An article of manufacture comprising cpn60 primers having the nucleotide sequences set forth in SEQ ID NO:12 and SEQ ID NO:13, and a cpn60 probe having the nucleotide sequence set forth in SEQ ID NO:18.

48. The article of manufacture of claim 47, further comprising instructions for using said cpn60 primers and probes to quantify the amount of C. jejuni in a biological or non-biological sample.

49. An article of manufacture comprising cpn60 primers having the nucleotide sequences set forth in SEQ ID NO:14 and SEQ ID NO:15, and a cpn60 probe having the nucleotide sequence set forth in SEQ ID NO:19.

50. The article of manufacture of claim 49, further comprising instructions for using said cpn60 primers and probes to quantify the amount of E. coil in a biological or non-biological sample.
